(19) United States
(12) Patent Application Publication
(10) Pub. No.: US 2004/0058314 A1
(43) Pub. Date: Mar. 25, 2004

(54) Assay for the detection and quantification of HBV cccDNA by real-time PCR

(76) Inventors: Ming Liang He, Hong Kong (CN); Hsiang-Fu Kung, Hong Kong (CN); Marie Chia Mi Lin, Hong Kong (CN)

Correspondence Address: WILLIAM COLLARD, COLLARD & ROE, P.C., 1077 NORTHERN BOULEVARD, ROSLYN, NY, 11576, US

(21) Appl. No.: 10/449,801

(22) Filed: May 29, 2003

Related U.S. Application Data
(60) Provisional application No. 60/383,953, filed on May 29, 2002.

(51) Int. Cl. C12Q 1/70; C12P 19/34

(52) U.S. Cl. 435/5; 435/91.2

(57) ABSTRACT

The persistence of covalently closed circular (ccc) DNA of Hepatitis B Virus (HBV) in liver cells is believed to be the major reason for relapse after completion of HBV antiviral therapy. Up to now, there is no sensitive method to quantify cccDNA in infected liver cells. A set of primers were designed to specifically amplify DNA fragments from HBV cccDNA but not from viral genomic DNA. A good linear range was obtained when 100 to 10⁷ copies of HBV cccDNA were used as template in the quantitative real-time PCR. Not only is this method rapid, economical, highly sensitive, it can be used to monitor HBV cccDNA in infected human liver biopsies and to guide patients undergoing long-term anti-HBV therapy.

### CROSS REFERENCE TO RELATED APPLICATIONS

This application is based upon provisional application serial No. 60/383,953 filed on May 29, 2002 wherein priority is claimed under 35 U.S.C. 119e.

### BACKGROUND

Chronic Hepatitis B virus (HBV) infection is one of the leading causes of death in the world. In Asia, more than 10% of the population is chronic infected by HBV See Seeger C. & Masson, W. S. (2000) Hepatitis B Virus Biology Micro Molec Biol Review 64, 51-58; and Orito E. et al (2001) Geographic Distribution of Hepatitis B Virus (HBV) genotype in patients with chronic HBV infection in Japan Hepatology 34, 590-594. See also He et al (2002) A New and Sensitive Method for the Quantification of HBV cccDNA by real time PCR, Academic Press: Biochemical and Biophysical Research Communications 295, 1102-1107 incorporated herein by reference. It is believed that HBV covalently closed circular DNA (cccDNA) contributes to the relapse after discontinuation of antiviral therapy See Yokosuka O. et al (1985) Changes of hepatitis B virus DNA in liver and serum caused by recombinant Leukocyte interferon treatment: analysis of intrahepatic replicative hepatitis B virus DNA, Hepatology 5, 728-734. To better treat this disease, it is necessary to monitor individual patients virologic profile in the antiviral therapy, especially the change of cccDNA level in the infected hepatocytes.

The replication of HBV undergoes an unusual intermediate form-cccDNA. In the viral particles, viral genome is partially double-stranded open circular DNA. Upon infection, the virus transports its DNA to the hepatocyte nucleus and converts it to a cccDNA using host cell enzymes. See Newbold, J. E. et al. (1995) The covalently closed duplex form of the hepadnavirus genome exists in situ as a heterogenous population of viral minochromosomes, Jornal of Virology 69, 3350-3357; see also Seeger and Masson (2000). Then the HBV cccDNA serves as the template to transcribe viral RNAs, which serve either as viral pregenome RNAs, or as mRNAs coding for the multifunctional polymerase, core, X and envelope (S) proteins. The pregenomic RNA is encapsulated by the virion core particle and reversely transcribed by the viral polymerase, forming a single-strand DNA (negative strand). There is a great probability to generate HBV variants at this step since the reverse transcriptase lacks proofreading activity. Summers J. and Mason W. S. (1982) Replication of the genome of a hepatitis B-like virus by reverse transcription of an RATA intermediate. Cell 29, 403-415; See also Seeger, C. & Masson, W. S. (2000).

Subsequently, the pregenome is degraded and the minus-strand DNA acts as a template for synthesis of a plus-strand DNA with variable length. Finally, HBV genome is either encapsulated to produce virions and be secreted out, or recycled back to the nucleus to maintain a pool of cccDNA molecules, resulting in the formation of a steady-state population of 5-50 ccc DNA molecules per infected hepatocyte see Tuttleman J. S. et al (1986) Formation of the pool of covalently closed circular viral DNA in hepadnavirus-infected cells, Cell 47, 451-460. All the HBV protiens play crucial roles in the HBV gene transcription, replication, viral packaging and recycling. Thus, monitoring HBV cccDNA level will provide more direct evidence to reflect the HBV activity in the body.

The HBV cccDNA pool in the nucleus is thought to be the major reason of antiviral drug resistance. It was shown that the cccDNA form was not eliminated in patients upon completion of antiviral therapy with interferon alpha, leading to a relapse See Yokosuka O. et al (1985). As HBV replication does not employ a semi-conservative mechanism, any nucleotide analogue-based therapy would not directly affect the pre-existing cccDNA template. This was confirmed in the animal model studies. See Dean, J. et al. (1995) Reversion of Duck Hepatitis B virus DNA replication in vivo following cessation of treatment with the nucleotide analogue ganciclovir, Antiviral Res 27, 171-178; See also Morelda, G. et al (1997) Lack of effect of antiviral therapy in nondividing hepatocyte cultures on the closed circular DNA of woodchuck hepatitis virus. J Virol 71, 9392-9399.

Accumulated evidence suggests that the eradication of chronic HBV infection require the elimination of the viral cccDNA from infected hepatocytes. See Locarni, S. A. et al (1996) Hepatitis B: New approaches for antiviral chemotherapy, Antiviral Chemistry and Chemotherapy 7, 1-12. It is important to develop a fast, sensitive method to measure cccDNA in the infected liver cells. The level of cccDNA in hepatocytes may be an important parameter in the decision of when to stop therapy in patients receiving long-term nucleoside analogue therapy.

Monitoring the level of cccDNA in the infected liver cells will provide useful information to the efficacy of treatment and long-term response. Quantification of HBV cccDNA by conventional techniques such as Southern hybridization is not sensitive enough because of the limited amount of clinical material available and the low copy number of cccDNA molecules in the liver cells. Recently, quantitative real-time PCR assay has successfully been used to monitor viral load in HBV, HCV, and HIV infections. Lewin, S. R. et al. (2001) Analysis of hepatitis B viral load decline under potent therapy: complex decay profiles observed. Hepatology 34, 1012-1020; Mitsunaga S. et al (2002) High-thoroughput HBV DNA and HCV RNA detection system using a nucleic acid purification robot and real time detection PCR; its application to analysis of posttransfusion hepatitis. Transfusion 42, 100-106; Cote, H. C. et al (2002) Changes in mitochondiral DNA as a marker of nucleoside toxicity in HIV-infected patients. N Engl J. of Med. 346, 811-820. It can accurately detect as low as 500 copies of virus in serum/ml, while the limitation for commonly used Digen Hybrid Capture II assay is 1.4×10⁵ viruses in serum/ml Lau, G. K. et al. (2002) High hepatitis B virus (HBV) DNA viral load as the most important risk factor for HBV reactivation in patients positive for HBV surface antigen undergoing autologous hematopoietic cell transplantation. Blood 99, 2324-2330. Since HBV viral DNA has a gap in the minus-strand and an incomplete plus-strand of variable length, selective cccDNA primers can be designed to amplify the region corresponding to the gap and incomplete region in the partially double-stranded virion DNA Littlejohn, M. et all (2001) Selective PCR for hepatitis B virus cccDNA: Validation fo real time assays. Hepatology 34, 311A.

### SUMMARY

The invention relates to a new method for the quantification of hepatitis B virus (HBV) of covalently closed circular (ccc) cccDNA by real time polymerase chain reaction or PCR. Pairs of primers to specifically amplify DNA fragments from HBV cccDNA but not the genomic DNA were used. A good linear range was observed from 100 to 10⁷ copies of cccDNA templates in a real-time PCR reaction. This new method provides a rapid and sensitive assay for quantification of cccDNA in infected hepatocytes, which will provide guidance for patients undergoing long-term anti-HBV therapy.

More specifically, the invention relates to a method for detecting covalently closed circular cccDNA of a Hepatitis B virus HBV in the form of a HBV cccDNA genome. The method starts by wherein a sample of liver cells infected with HBV cccDNA virus is obtained from a patient. Next, at least one primer is prepared for applying to at least one end of the HBV cccDNA virus. Next, the HBV cccDNA virus is amplified by a polymerase chain reaction (PCR) using the at least one primer. Next, at least one probe is prepared for applying to the HBV cccDNA gnome, wherein the probe comprises a dye and a dye quencher. Next, a second polymerase chain reaction is conducted to bind the at least one primer to the at least one probe so that the dye and the dye quencher in the probe are separated allowing the HBV cccDNA to be detected via the dye.

The sequence of the HBV can be any full-length HBV genomic sequence in the GeneBank. The HBV sequence used was published in Science in China in 1986 and is also listed as (SEQ ID NO. 11). The plasmid information can also obtained from a published paper (Fu, L., Wu, X., Kong, Y. & Wang, Y. Regulation of HBV gene expression by core promoter and its upstream sequence. Chin J. Virol., 13, 215-223, 1997).

With respect to the sequences, in a DNA sequence, B means nucleotide C, G, or T; W means nucleotide A or T.

Essentially the invention relates to a method for detecting covalently closed circular cccDNA of a Hepatitis B virus HBV in the form of a HBV cccDNA genome. The method can contain the following steps; obtaining a sample of liver cells infected with HBV cccDNA virus from a patient. Next, the process involves preparing at least one primer for applying to at least one end of the HBV cccDNA virus. The next step includes amplifying the HBV cccDNA virus by a polymerase chain reaction (PCR) using the at least one primer. Next, the process involves preparing at least one probe for applying to the HBV cccDNA gnome, wherein the probe comprises a dye and a dye quencher. The next step involves conducting a second polymerase chain reaction to bind the at least one primer to the at least one probe so that the dye and the dye quencher in the probe are separated allowing the HBV cccDNA to be detected via the dye.

The invention also relates to a kit comprising for the detection of HBV cccDNA in a patient comprising a plurality of forward amplification primers for detecting cccDNA wherein the forward primers consist of: (5′-(SEQ ID NO: 1)-3′) (5′-(SEQ ID NO: 2)-3′) (5′-(SEQ ID NO: 3)-3′). There are also a plurality of reverse primers wherein these reverse primers consist of: (5′ (SEQ ID NO: 4)-3′); (5′(SEQ ID NO: 5)-3′); and (5′ (SEQ ID NO: 6)-3′). This kit is used to perform the steps described above.

### BRIEF DESCRIPTION OF THE DRAWINGS

Other objects and features of the present invention will become apparent from the following detailed description considered in connection with the accompanying drawings, which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 shows the structure of the HBV genome. The HBV genome is composed of partial single-stranded, partial double-stranded and partial triple-stranded DNA in the viral particle;

FIG. 2A is the diagram of detection of HBV cccDNA wherein HBV cccDNA can be detected by real-time PCR;

FIG. 2B is a diagram of detection for HBV cccDNA wherein HBV genomic DNA cannot be detected by real-time PCR;

FIG. 3 is a photo of a primer selection wherein PCR was performed to select primer pairs which can specifically amplify a DNA fragment from HBV cccDNA but not genomic DNA in the PCR reaction;

FIG. 4 shows the real-time PCR profile of primer pairs using 10⁶ copies of cccDNA as template;

FIG. 5A shows a real-time PCR performance of primer pair HBV-CCC-F1/HBV-CCC-R1 when 100 to 10⁷ copies of HBV cccDNA were used as template;

FIG. 5B shows a standard curve wherein a good linear range was obtained when 100 to 10⁷ copies of HBV cccDNA were used as template;

FIG. 6 shows a graph of real-time PCR which can quantitatively differentiate HBV genomic DNA from replication intermediate DNA;

FIG. 7A shows a table of a reading of a first patient; and

FIG. 7B shows a table of a reading of a second patient.

### DETAILED DESCRIPTION

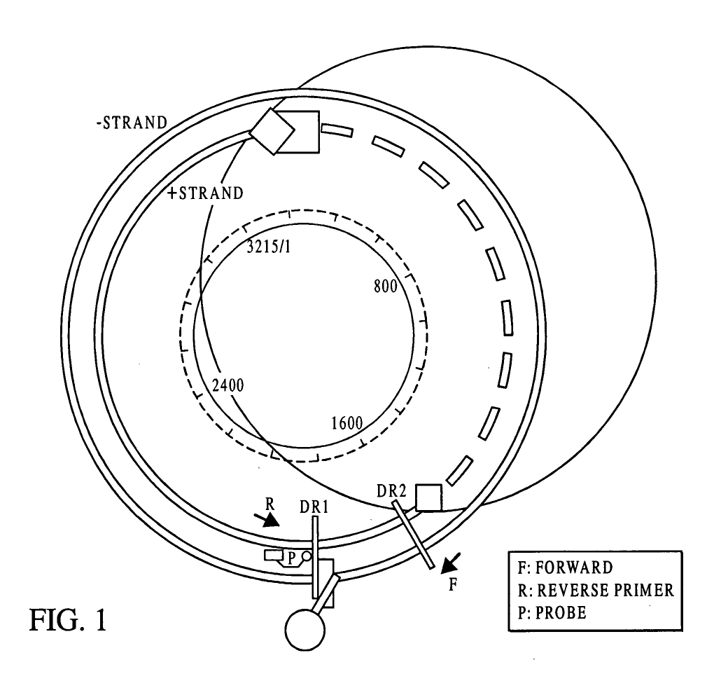

Referring in detail to the drawings, FIG. 1 shows the structure of the HBV genome. The HBV genome is composed of partial single-stranded, partial double-stranded and partial triple-stranded DNA in the viral particle. The length of the plus strand is variable, but the negative strand has a definite 5′ end and 3′ end near position nucleotide 1800. The principle of quantitative measurement of HBV cccDNA occurs wherein in an HBV genome, the incomplete plus strand has a variable 3′ end but a defined 5′ end around position 1600 near DR2, while the complete minus strand has defined 5′ and 3′ ends with a terminal redundancy of 9 bases. See Zuckerman, A. J. et al Viral Hepatitis Harcourt Publishers Ltd Press, USA 1998. There is a gap around position 1800 near DR1 as shown in FIG. 1. Therefore, primers can be designed and selected to specifically amplify DNA fragments from replication intermediate cccDNA but not from viral genome DNA. As shown in FIG. 1, the circle shows the primase while the diamond shape shows the DNA polymerase. In this case, both strands are closed with no overhang.

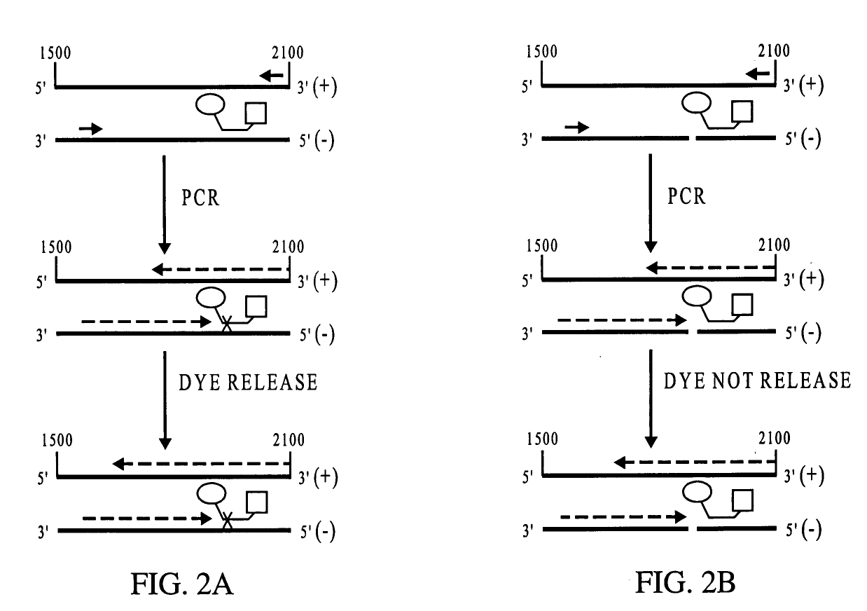

The primers and probe are applied to the cccDNa simultaneously during the real time PCR. However, the probe cannot become a new strand in the PCR reaction because the 3′-end of the probe is blocked by the quencher linked at the 3′ end. As shown in FIG. 1, the Taq DNA polymerase will extend the primers and become a new strand during the PCR reaction. The TaqDNA will finally meet and cut the probe, and the dye gets released and detected by the machine when the cccDNA is used as a template as shown in FIG. 2A. However, the dye will not be released and detected by a machine when HBV genomic DNA was used as a template because a chain extension cannot go through the gap to meet the probe as shown in FIG. 2B.

FIG. 2A and FIG. 2B highlight the difference surrounding the detection of HBV cccDNA vs HBV genomic DNA. For example, FIG. 2A is the diagram of detection of HBV cccDNA, wherein HBV cccDNA can be detected by real-time PCR. In contrast, FIG. 2B is a diagram of detection for HBV cccDNA wherein HBV genomic DNA cannot be detected by real-time PCR.

During this process, a TaqMan probe is used for detection of ccc DNA. This TaqMan probe binds to DNA when PCR is performed, and is composed of dye and dye quencher. As shown in FIG. 2A, the fluorescence signals are released and detected by ABI 7900 Sequence detector when Taq DNA polymerase meet and cut the probe to separate the dye and quencher. In contrast, as shown in FIG. 2B, PCR cannot proceed properly if viral genome DNA is used as a template because the dye and quencher are not separated. To reduce the background, the primer was designed to bind the negative strand on one side of the gap, and the probe to bind the same strand on the other side. As shown in FIG. 2B, Taq DNA polymerase will not be able to reach the probe if the viral genome DNA is used as template in a real-time PCR reaction. In this case, no signal can be detected no matter how the plus strand is amplified in the reaction.

Thus, to detect the presence of HBV cccDNA, sets of primers that specifically amplify HBV DNA from cccDNA but not from virus genome DNA were created. Over 150 known sequences of HBV were aligned in GenBank, including all the genotypes (A to G). According to the sequence conservation, three forward primers (HBV-CCC-F1, HBV-CCC-F2, HBV-CCC-F3) and three reverse primers (HBV-CCC-R1, HBV-CCC-R2 and HBV-CCC-R3) were designed. To cover different genotypes, some primers were degenerated. Different primer combinations were used in the primer selection PCR experiments.

Both cccDNA and viral genome DNA were used as templates for primer selection. The cccDNA was obtained by isolation of supercoil plasmid containing the full-length HBV genome and its concentration was determined measurement of OD260 and verified by agarose gel electrophoresis. See Fu, L. et al. (1997) Regulation of HBV gene expression by core promoter and its upstream sequence. Chinese J. Virology 13, 216-223; Ausubel, F. et al, ed (1998) Current Protocols in Molecular Biology, John Wiley & Sons, Inc.). The viral genomic DNA was obtained from a derivative of HepG2 cell line (HepAD38), which constantly produces HBV viral particles in the medium without tetracycline See Ladner, S. K. et al. (1999) The hepatitis B virus M539V polymerase variation responsible for 3TC resistance also confers cross resistance to other nucleoside analogues. Antivir Chem Chemother. 9, 65-72. The copy number of the isolated genome was determined by real-time PCR using a commercial HBV detection kit (PG Biotech. Com., Ltd., China).

The 10⁶ copies of plasmid DNA or 1.6×10⁶ copies of viral DNA were used as templates. The results showed that four primer combinations (HBV-CCC-F1/HBV-CCC-R1, HBV-CCC-F1/HBV-CCC-R3, HBV-CCC-F3/HBV-CCC -R1, and HBV-CCC-F3/HBV-CCC-R2) gave rise to specific band from plasmid template samples while no band from viral genome DNA samples after PCR amplification. All the combinations containing HBV-CCC-F2 gave rise to some background. In addition, the primer HBV-CCC-F3/HBV-CCC-R3 combination also gave a smear band for unknown reasons.

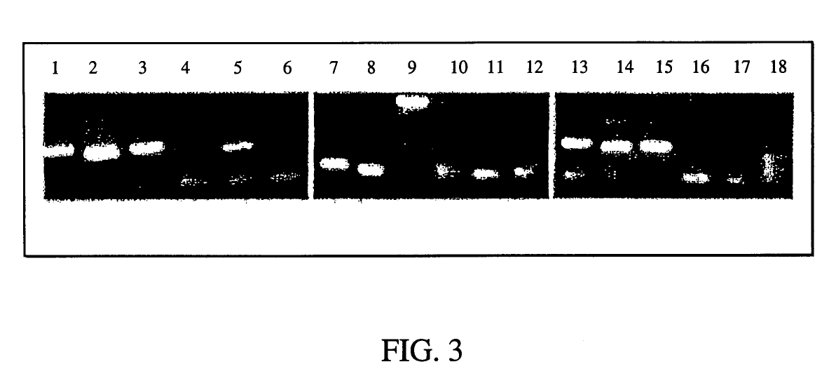

For example, FIG. 3 shows a photo of a primer selection wherein PCR was performed to select primer pairs which can specifically amplify DNA fragment from HBV cccDNA but not genomic DNA in the PCR reaction. 10⁶ copies of cccDNA from a plasmid containing HBV genome were used as template in lane 1, 2, 3, 7, 8, 9, 13, 14 and 15; while 1.6×10⁶ copies of virus DNA isolated from AD38 condition medium were used as template in lane 4, 5, 6, 10, 11, 12, 17, 18, 19. The primer pairs that were used were: 1, 4, HBV-CCC-F1/HBV-CCC-R1; 2, 5, HBV-CCC-F1/HBV-CCC-R2; 3, 6, HBV-CCC-F1/HBV-CCC-R3; 7, 10, HBV-CCC-F2/HBV-CCC-R1; 8, 11, HBV-CCC-F2/HBV-CCC-R2 9, 12, HBV-CCC-F2/HBV-CCC-R3; 13, 16, HBV-CCC-F3/HBV-CCC-R1; 14, 17, HBV-CCC-F3/HBV-CCC-R2; 15, 18, HBV-CCC-F3/HBV-CCC-R3.

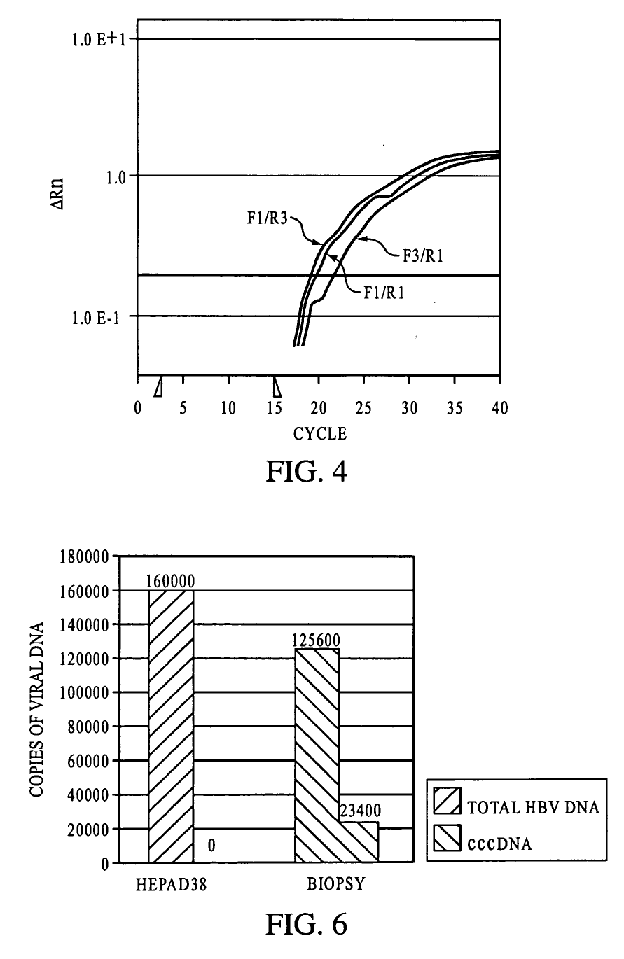

FIG. 4 shows the real-time PCR profile of primer pairs using 10⁶ copies of cccDNA as template, wherein F1 is HBV-CCC-F1; while R1 is HBV-CCC-R1, while R3 is HBV-CCC-R3. Real-time PCR can accurately measure cccDNA of HBV. Thus, the primer pairs with good specificity in the PCR experiments were used to test the performance in the real-time PCR reaction. The results showed that HBV-CCC-F1/HBV-CCC-R1, HBV-CCC-F1/HBV-CCC-R3 and HBV-CCC-F3/HBV-CCC-R1 gave satisfactory performance when 10⁶ copies of supercoil plasmid were used as templates.

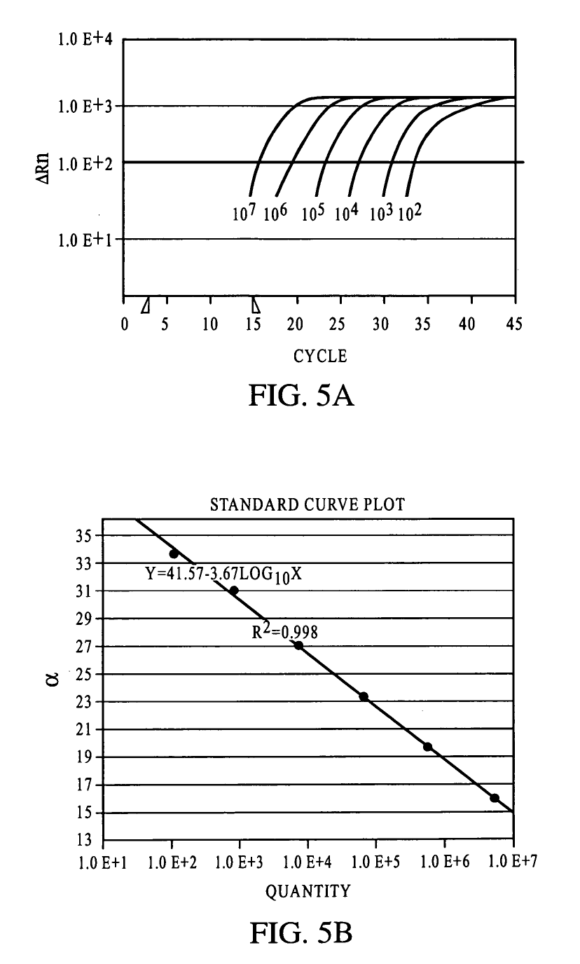

In addition, HBV-CCC-F1/HBV-CCC-R1 primer pair was chosen for further experiments wherein FIG. 5A shows a real-time PCR performance of primer pair HBV-CCC-F1/HBV-CCC-R1 when 100 to 10⁷ copies of HBV cccDNA were used as template. These results showed a good linear range when 100 to 10⁷ copies of plasmid DNA were used as templates. Thus, FIG. 5B shows a standard curve wherein a good linear range was obtained when 100 to 10⁷ copies of HBV cccDNA were used as template.

To confirm whether the HBV-CCC-F1/HBV-CCC-R1 primer pair can specifically and quantitatively differentiate cccDNA and genomic DNA, real-time PCR experiments were performed using HBV DNA isolated from HepAD38 conditioned medium or patient liver biopsy as templates for cccDNA detection. The result showed that no signal was detected using DNA templates isolated from HepAD38 conditioned medium, while 2.3×10⁴ copies of cccDNA were detected using DNA templates isolated from patient liver biopsy Thus, FIG. 6 shows a graph of real-time PCR which can quantitatively differentiate HBV genomic DNA from replication intermediate DNA.

Clinically, the cccDNA level of HBV in infected hepatocytes may be an important parameter for antiviral therapy. A set of primers can specifically amplify replication intermediate cccDNA of HBV but not genomic DNA. These primers gave rise to a good linear range in the quantitative real-time PCR reaction when 100 to 10⁷ copies of HBV cccDNA were used as templates, while zero background was observed using HBV viral genome DNA as template.

The quantitative measurement of cccDNA in liver is not fully developed due to the lack of primer specificity to cccDNA and genomic DNA. The selectivity of the HBV cccDNA primers is not absolute; partial elongation products can be produced from the PCR of non-ccc forms of HBV DNA along with high levels of background. To overcome this problem, the design of the primers and probe involved a unique strategy. In the assays, the probe could not be cut by Taq DNA polymerase to release the signal using genomic DNA templates. Therefore, this assay can perfectly differentiate between the genomic DNA and cccDNA as shown by the results in FIG. 6.

The primers and probe may also be used to quantify HBV cccDNA with multiple genotypes. To overcome the difference among the sequences, over 150 known sequences in the GenBank and designed degenerated probe and primers. Blast assay (NCBI) showed that HBV-CCC-F1 fits genotype A (GenBank accession. No. AJ309371, AJ309370), genotype B (No. AF121250), genotype C (No. AF461358), and genotype A/D (No.AF297620). Primer HBV-CCC-R1 also fits multiple genotypes, including genotype A, genotype B, genotype C, genotype G (No.AF3-63962) and genotype F (No.AB036920). This allows our assay to cover over 90% of patients in the Asia-pacific area.

This novel quantitative real-time PCR assay for HBV cccDNA will provide a powerful tool for clinical studies. The results from the studies on cccDNA kinetics of infected hepatocytes should provide useful guidance for patients undergoing long-terms anti-HBV therapy.

Experimental Protocol: Experiment 1

cccDNA preparation: Chinese HBV DNA (genotype C) cloned in pBS vector (p3.6II) was a gift from Professor Yuan Wang (16). The supercold plasmid (cccDNA) was isolated by CsCl purification Ausubel, F. et al, ed (1998) Current Protocols in Molecular Biology, John Wiley & Sons, Inc.

Cell culture and HBV viral DNA isolation: HepAd38 cells were obtained from Professor Stephen Locarnini and were cultured at 37 C in DMEM medium supplemented with 10% (v/v) fetal bovine serum, 100 IU/ml penicillin/streptomycin, 500:g/ml G418, and 1:g/ml tetracycline. To obtain HBV viral DNA, HepAD38 cells were seeded in 12-well culture plates. After three days, the medium was removed and replaced by fresh medium lacking tetracycline. Viral genome DNA was extracted from either AD38 conditioned medium after another two days of culture or a patient liver biopsy (HBeAg+) using a QIAamp DNA Blood Mini Kit (QIAGEN, Hilden, Germany) according to the manufacturers instructions.

Primers and PCR: Several pairs of primers were designed to amplify HBV DNA fragment using p3.6II supercoid plasmid or HBV genome DNA as templates. The forward primers were HBV-CCC-F1: 5′-(SEQ ID NO: 1)-3′; HBV-CCC-F2: 5′-(SEQ ID NO: 2)-3′; HBV-CCC-F3: 5′-(SEQ ID NO: 3)-3′; the reverse primers were HBV-CCC-R1: 5′-(SEQ ID NO: 4)-3′; HBV-CCC-R2: 5′-(SEQ ID NO: 5)-3′; HBV-CCC-R3: 5′-(SEQ ID NO: 6)-3′. The PCR primers and probe are conserved among over 150 known sequence obtained from GenBank corresponding to different genotypes. PCR was performed using combined primer pairs under the following conditions: 94×2 min, [95×15 sec—54×30 sec—72×1 min]×40 cycles, 72 5 min.

Quantification of HBV viral DNA and cccDNA using Real-time PCR: For HBV DNA quantification by real-time PCR, PCR amplification was performed with a set of PCR primers and a probe, which covers the HBV genome from nucleotide 1500-2100, corresponding to the surface antigene gene (S gene) of the HBV. The PCR primers were: HBV-S-F: 5′-(SEQ ID NO: 7)-3′, HBV-S-R: 5′-(SEQ ID NO: 8)-3′. The TaqMan probe was 5′-TET-(SEQ ID NO: 9)-TAMRA-3′. The PCR were performed according to the manufacture instructions (PG Biotech. Ltd, China) using ABI 7900HT Sequence Detection System. For quantification of HBV cccDNA by real-time PCR, 250 nM of the probe (5′-FAM-(SEQ ID NO: 12)-TAMRA-3′ and 900 nM of the two PCR primers were used. FAM is the fluorescence die linked at the 5′ end, and TAMRA is a quencher linked at the 3′ end. As shown in FIG. 1, the Taq DNA polymerase will extend the primers and become a new strand during the PCR reaction. The PCR reaction was carried out in a 20:1 volume using Master Mix (ABI, USA). The PCR cycling program consisted of an initial denaturing step at 95° C. for 10 min, followed by 45 amplification cycles at 95° C. for 15 sec., 61.5° C. for 1 min. Essentially, the Taq DNA polymerase will finally meet and cut the probe and the dye gets released and detected by the machine when cccDNA is used as a template as shown in FIG. 2A. However, the dye will not be released and detected by a machine when HBV genomic DNA was used as a template because the chain extension cannot go through the gap to meet the probe as shown in FIG. 2B.

Experimental Protocol: Experiment 2

Materials and Methods

cccDNA standard: A plasmid containing Chinese HBV genome (PHBV-adr) was a gift from Professor Yuan Wang. The supercoid plasmid (cccDNA) was isolated by CsCl purification. The cccDNA concentration was determined by measurement of OD260 and verified by agarose gel electrophoresis. The copy number was determined by its molecule weight.

HBV viral DNA preparation: HBV viral DNA was extracted from either 200 ul of patient sera or weighted liver biopsies using a QIAamp DNA Blood or Tissue Mini Kit (QIAGEN, Hilden, Germany) according to the manufacturer's instructions.

Quantification of HBV cccDNA using Real-time PCR: The method for HBV cccDNA and genomic DNA quantification was described by He et al. (BBRC., 2002) with minimor modification. Briefly, a pair of primers (forward primer: 5′-(SEQ ID NO: 1) 3′, reverse primers 5′-(SEQ ID NO: 4)-3′), which can specifically amplify a DNA fragment from HBV cccDNA, but not viral genomic DNA by PCR, were used for real-time PCR. In a typical real-time PCR reaction, 250 nM of the probe (5′-FAM(SEQ ID NO:10)-3′) and 900 nM of the two PCR primers were used. For total HBV quantification, PCR amplification was performed with a HBV DNA diagnostic kit (PG Biotech. Ltd, China) using ABI 7900HT Sequence Detection System. The PCR program consisted of an initial denaturing step at 95° C. for 10 min, followed by 40 amplification cycles at 95° C. for 15 sec, 61.5° C. for 1 min.

Patients samples: The patients were treated with chemotherapy at Queen Mary Hospital, Hong Kong SAR China from Jan. 2000 to May 2002. In accordance with the standard protocols, all patients who received chemotherapy were screened for HBsAg, HBsAb, human immunodeficiency virus antibody (HIV Ab), HBV DNA by PCR and hepatitis C antibody (anti-HCV) using commercially available enzyme immunoassays (Abbott Laboratories, Chicago, Ill., U.S.A.) and HBcAb was tested by RIA (Corab; Abbott). For all HBsAg positive patients, further serological testing for hepatitis B e antigen (HBeAg), hepatitis B e antibody (HBeAb) and serum HBV DNA by PCR and were performed chemotherapy with lamivudine. All these HBsAg positive recipients were tested at 2-weekly interval for liver function tests (including serum alanine aminotransferase, serum albumin and bilirubin) and serum HBV DNA during chemotherapy. Hepatitis serology (HBsAg, HBeAg, HBeAb, HBV DNA by PCR, and HCV RNA by RT-PCR) were performed on the serum collected preceding and during the events whenever there was any clinical suspicion of liver damage due to hepatitis B infection. The occurrence of hepatic events (acute hepatitis, chronic hepatitis, anicteric and icteric hepatitis, hepatic failure) were recorded. Hepatitis was defined as a more than three-fold elevation of serum aminotransferase above the upper limit of normal, on two consecutive determinations at least five days apart. HBV reactivation was defined when preceded or accompanied by an elevation of serum HBV DNA to more than ten times that of the pre-exacerbation baseline, the serum HBV DNA turned from negative to positive, or the HBsAg became positive and remained so for two consecutive readings five days apart.

All serum and biopsy samples were stored at −70° C. All patients who had post-chemotherapy hepatitis due to HBV reactivation were treated with lamivudine 100 mg once daily.

Result:

The HBV cccDNA exists in all the patients' hepatocytes but only in a subset of patients' sera. To elucidate the cccDNA status in the HBV patients, the cccDNA and total HBV DNA level was quantified in the liver biopsies. The total DNA was isolated from weighted liver biopsies and quantified with a HBV diagnostic kit by real-time PCR. The HBV cccDNA was also measured by real-time PCR. Our results showed that the HBV cccDNA was detected in all the HBV patients' liver biopsies. The copy number of cccDNA in the patient hepatocytes is from 0.05 to 168 per cell. The copy number of the total HBV DNA in the patient hepatocytes is from 0.08 to over 3000 copies per cell. The ratio of the cccDNA to the total HBV DNA is from 1 to 10,000 (Table 1), indicating that the cccDNA in patient hepatocytes have active replication and relative silent status. The HBV cccDNA may also be in the patients' sera because of liver inflammation and necrosis. To test this hypothesis, the HBV DNA was isolated from patients' sera and there was performed quantitative PCR. The results showed that the cccDNA could only be detected in a subset of patients' sera though the HBV DNA was detected in all the patients' sera (Table 1). The ratio of the cccDNA to the total HBV DNA is 0 to 1.7%.

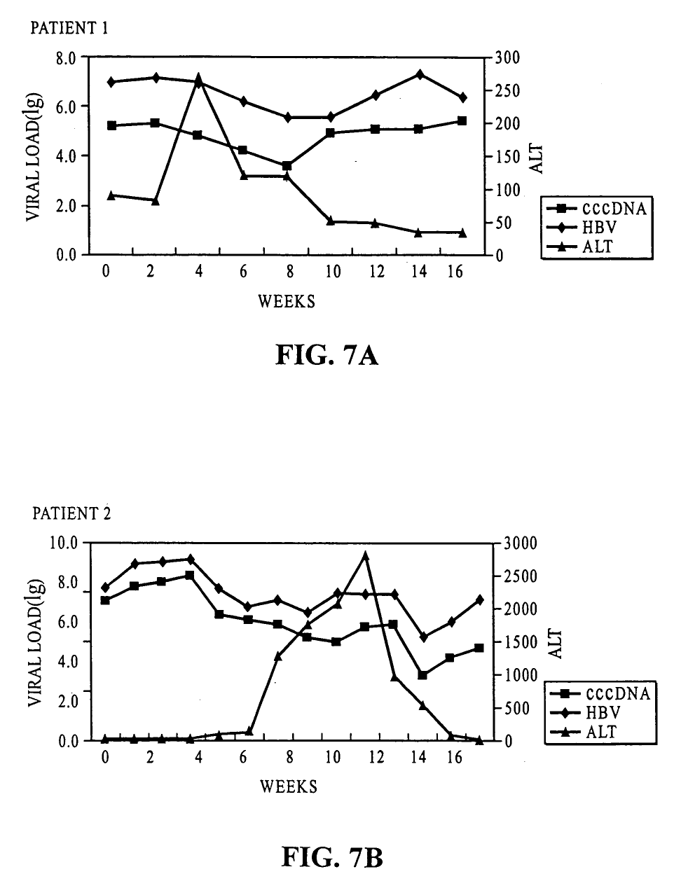

Conclusion: The cccDNA is an early signal of liver damage To investigate the medical significance of the cccDNA in sera, longitudinal studies were performed. Patients with or without HBV reactivation during lamivudine treatment were chosen for this study. The patients' sera were collected every two weeks and the HBV DNA was isolated for the quantification of the HBV cccDNA and total HBV DNA. The results showed that the HBV cccDNA was not detectable by sensitive real-time PCR in the patients without the HBV reactivation. However, the cccDNA was detected in the patients with the HBV reactivation (FIGS. 7A and 7B). The cccDNA level was correlated with viral load, which occurred earlier than the ALT. Before the ALT value increased, the cccDNA level and viral load had elevated to a high level. With the ALT increases, the cccDNA level dropped rapidly. These results suggested that the patients with cccDNA in sera have high risk for HBV reactivation, and the occurrence of the HBV cccDNA in the sera comes earlier than the ALT elavation.

The results suggest that the cccDNA level in the sera may be an important parameter for anti-HBV treatment, and low level of cccDNA in hepatocytes be an end point of treatment.

TABLE 1
HBV cccDNA in the patients' hepatocytes and sera

| | HBV DNA in the Biopsies | | | HBV DNA in the sera | | |
|---|---|---|---|---|---|---|
| | (copy/cell) | | | (copy/ml) | | |
| | | HBV | HBV/ | | HBV | ccc/ |
| Patients | cccDNA | DNA^{a} | ccc^{b} | cccDNA | DNA^{c} | HBV^{d} |
| 1 | 2.05 | 195.5 | 98.7 | 3.4 × 10⁶ | 4.8 × 10¹⁰ | 0.01% |
| 2 | 0.03 | 0.17 | 5.8 | UN | 6.6 × 10⁷ | — |
| 3 | 173.1 | 799.5 | 4.6 | 3.1 × 10⁵ | 6.0 × 10¹⁰ | 0.001% |
| 4 | 0.16 | 8.13 | 51.7 | UN | 1.9 × 10⁷ | — |
| 5 | 0.03 | 0.08 | 2.8 | UN | 5.4 × 10⁵ | — |
| 6 | 16.02 | 17.4 | 1.1 | 2.4 × 10⁶ | 5.3 × 10⁸ | 0.46% |
| 7 | 18.03 | 3717 | 206 | 3.1 × 10⁷ | 1.8 × 10¹¹ | 0.02% |
| 8 | 0.79 | 13.1 | 16.5 | UN | 2.4 × 10¹⁰ | — |
| 9 | 0.05 | 153.3 | 3406 | 2.8 × 10⁵ | 3.0 × 10⁹ | 0.01% |
| 10 | 2.06 | 16.4 | 8 | 2.7 × 10⁶ | 7.5 × 10⁸ | 1.77% |

Legend:
^{a}The total HBV copies per cell in the patient's liver biopsies;
^{b}The ratio of total HBV DNA to cccDNA in the patient's liver biopsy;
^{c}The total HBV copies in serium per ml in the patient's sera; and
^{d}The ratio of cccDNA to total HBV DNA in the patient's sera.

Accordingly, while at least one embodiment of the present invention has been shown and described, it is to be understood that many changes and modifications may be made thereunto without departing from the spirit and scope of the invention as defined in the appended claims.

What is claimed is:

1. A method for detecting covalently closed circular cccDNA of a Hepatitis B virus HBV in the form of a HBV cccDNA genome, the method comprising the steps of:
a) obtaining a sample of liver cells infected with HBV cccDNA virus from a patient;
b) preparing at least one primer for applying to at least one end of the HBV cccDNA virus;
c) amplifying said HBV cccDNA virus by a polymerase chain reaction (PCR) using said at least one primer;
d) preparing at least one probe for applying to said HBV cccDNA gnome, wherein said probe comprises a dye and a dye quencher; and
e) conducting a second polymerase chain reaction to bind said at least one primer to said at least one probe so that said dye and said dye quencher in said probe are separated allowing said HBV cccDNA to be detected via said dye.

2. The process as in claim 1, wherein said step of preparing said at least one primer comprises preparing at least one forward primer.

3. The process as in claim 1, wherein said step of preparing said at least one primer comprises preparing at least one reverse primer.

4. The process as in claim 1, wherein said step of preparing at least one primer comprises preparing at least one primer pair including at least one forward primer and at least one reverse primer.

5. The process as in claim 4, wherein said step of preparing at least one primer includes using between 100 and 10⁶ copies of plasmid HBV cccDNA as a template.

6. The process as in claim 4, wherein said step of preparing at least one primer includes using between 100 and 10⁷ copies of plasmid HBV cccDNA as a template.

7. The process as in claim 1 wherein said step of preparing at least one primer comprises preparing at least one primer pair including at least three different forward primers and at least three different reverse primers.

8. The process as in claim 1, wherein said step of preparing at least one primer includes preparing a primer consisting of: (5′-(SEQ ID NO: 1)-3′)

9. The process as in claim 1, wherein said step of preparing at least one primer includes preparing a primer consisting of: (5′-(SEQ ID NO: 2)-3′).

10. The process as in claim 1, wherein said step of preparing at least one primer includes preparing a primer consisting of: (5′-(SEQ ID NO: 3 -3′).

11. The process as in claim 1, wherein said step of preparing at least one primer includes preparing a primer consisting of: (5′-(SEQ ID NO: 4) -3′).

12. The process as in claim 1, wherein said step of preparing at least one primer includes preparing a primer consisting of: (5′-(SEQ ID NO: 5) -3′).

13. The process as in claim 1, wherein said step of preparing at least one primer includes preparing a primer consisting of: (5′-(SEQ ID NO: 6) -3′).

14. The process as in claim 1, further comprising the step of:
quantifying a presence of both HBV viral and HBV cccDNA wherein said primer and said probe correspond to a surface antigene (Sgene) of said HBV.

15. The process as in claim 14, wherein said step of preparing at least one primer includes preparing a primer consisting of: (5′-(SEQ ID NO: 7) -3′).

16. The process as in claim 14, wherein said step of preparing at least one primer includes preparing a primer consisting of: 5′-(SEQ ID NO: 8)-3′.

17. The process-as in claim 14, wherein said step of preparing at least one probe includes preparing a probe consisting of: (5′-TET-(SEQ ID NO: 9)- TAMRA-3′).

18. The process as in claim 14, wherein said step of preparing at least one probe includes preparing a probe consisting of: (5′-FAM-(SEQ ID NO: 10)-TAMRA-3′).

19. A kit for the detection of HBV cccDNA in a patient comprising:
a) a plurality of forward amplification primers for detecting cccDNA wherein said forward primers consist of: (5′-(SEQ ID NO: 1) -3′); (5′-(SEQ ID NO: 2) -3′); (5′-(SEQ ID NO: 3)-3′); and
b) a plurality of reverse primers wherein said reverse primers consist of: (5′-(SEQ ID NO: 4)-3′); (5′-(SEQ ID NO: 5)-3′); (5′-(SEQ ID NO: 6)-3′).

20. The kit as in claim 19, further comprising at least one probe.

21. The kit as in claim 20, wherein said probe comprises: 5′-TET-(SEQ ID NO: 9)-TAMRA-3′.

22. The kit as in claim 20, wherein said probe comprises: (5′-FAM-(SEQ ID NO: 12) -TAMRA-3′).


SEQUENCE LISTING (12 sequences)

SEQ ID NO: 1 - DNA, 21 bp, Artificial sequence
  synthetic DNA
actcttggactcbcagcaatg

SEQ ID NO: 2 - DNA, 18 bp, Artificial Sequence
  synthetic DNA
tgttcaccagcaccatgc

SEQ ID NO: 3 - DNA, 20 bp, Artificial sequence
  synthetic DNA
gcggwctccccgtctgtgcc

SEQ ID NO: 4 - DNA, 21 bp, Artificial sequence
  synthetic DNA
ctttatacgggtcaatgtcca

SEQ ID NO: 5 - DNA, 21 bp, Artificial sequence
  synthetic DNA
acagcttggaggcttgaacag

SEQ ID NO: 6 - DNA, 20 bp, Artificial sequence
  synthetic DNA
gtccatgccccaaagccacc

SEQ ID NO: 7 - DNA, 18 bp, Artificial sequence
  synthetic DNA
cctcttcatcctgctgct

SEQ ID NO: 8 - DNA, 19 bp, Artificial sequence
  synthetic DNA
aactgaaagccaaacagtg

SEQ ID NO: 9 - DNA, 27 bp, Artificial sequence
  synthetic DNA
tcccatcccatcatcctgctgggcttt

SEQ ID NO: 10 - DNA, 33 bp, Artificial sequence
  synthetic DNA
ctttttcacctctgcctaatcatctcwtgttca

SEQ ID NO: 11 - DNA, 3215 bp, Hepatitis B virus
aactccacaacattccaccaagctctgctagaccccagagtgaggggcctatactttcctgctggtggctccagttccggaacagtaaaccctgttccgactactgcctcacccatatcgtcaatcttctcgaggactggggaccctgcaccgaacatggagagcacaacatcaggattcctaggacccctgctcgtgttacaggcggggtttttcttgttgacaagaatcctcacaataccacagagtctagactcgtggtggacttctctcaattttctagggggagcacccacgtgtcttggccaaaattcgcagtccccaacctccaatcactcaccaacctcttgtcctccaatttgtcctggttatcgttggatgtgtctgcggcgttttatcatattcctcttcatcctgctgctatgcctcatcttcttgttggttcttctggactaccaaggtatgttgcccgtttgtcctctacttccaagaacatcaactaccagcacgggaccatgcaagacctgcacgattcctgctcaaggaacctctatgtttccctcttgttgctgtacaaaaccttcggacggaaactgcacttgtattcccatcccatcatcttgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccccaataaaaccaaacgttggggctattcccttaatttcatgggatatgtaattggatgttggggtactttaccgcaagaacatattgtactaaaaatcaagcaatgttttcgaaaactgcctgtaaatagacctattgattggaaagtatgtcagagaattgtgggtcttttgggctttgctgccccttttacacaatgtggctatcctgccttgatgcctttatatgcatgtatacaatctaagcaagctttcactttctcgccaacttacaaggcctttctgtgtaaacaatatctgaacctttaccccgttgcccggcaacggtcaggtctctgccaagtgtttgctgacgcaacccccactggatggggcttggctattggccatcgccgcatgcgtggaacctttgtggctcctctgccgatccatactgcggaactcctggcagcctgttttgctcgcagccggtctggagcaaaacttatcggaaccgacaactctgttgtcctctctcggaaatacacctcctttccatggctgctcgggtgtgctgccaactggatcctgcgcgggacgtcctttgtctacgtcccgtcggcgctgaatcccgcggacgacccgtctcggggccgtttgggcctctatcgtccccttcttcatctaccgttccggccgaccacggggcgcacctctctttacgcggtctccccgtctgtgccttctcatctgccggaccgtgtgcacttcgcttcacctctgcacgtcgcatggagaccaccgtgaacgcccaccaggtcttgcccaaggtcttacataagaggactcttggactcgcagcaatgtcaacgaccgaccttgaggcatacttcaaagactgtttgtttaaggactgggaggagttgggggaggagattaggttaaaggtctttgtactgggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctcagctctgtatcgggaggccttagagtctccggaacattgctcacctcaccataccgcactcaggcaagctattctgtgttggggtgagttgatgaatccggccacctgggtgggaagtaatttggaagacccagcatccagggaattagtagtcagctatgtcaatgttaatatgggcctaaaaatcagacaactactgtggtttcacatttcctgtcttacttttggaagagaaactgttcttgagtatttggtgtcttttggagtgtggattcgcactcctcctgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctatctttaatcctgagtggcaaactccctcctttcctcacattcatttacaggaggacattattaatagatgtcaacaatatgtgggccctcttacagttaatgaaaaaaggagattaaagttaattatgcctgctaggttctatcctaaccttaccaaatatctgcccttggacaaaggcattaaaccatattatcctgaacatgcagttaatcattacttcaaaactaggcattatttacatactctgtggaaggcgggcattctatataagagagaaactacgcgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcagggcatattgacaacagtgccagtagcacctcctcctgcctccaccaatcggcagtcaggaagacagcctactcccatctctccacctctaagagacagtcatcctcaggccatgcagtgg

SEQ ID NO: 12 - DNA, 25 bp, Hepatitis B virus
ttcaagcctccaagctgtgcctttg